(19) United States
(12) Patent Application Publication
(10) Pub. No.: US 2023/0132440 A1
(43) Pub. Date: May 4, 2023

(54) EXTRACORPOREAL TREATMENT OF COVID-19

(71) Applicant: MARV ENTERPRISES LLC, HERMITAGE, PA (US)

(72) Inventor: Mitchell S. Felder, Hermitage, PA (US)

(21) Appl. No.: 17/911,895

(22) PCT Filed: Mar. 16, 2021
(86) PCT No.: PCT/US2021/022541

§ 371 Date: Sep. 15, 2022

Related U.S. Application Data
(60) Provisional application No. 62/989,981, filed on Mar. 16, 2020.

(51) Int. Cl. A61M 1/36; A61M 1/34

(52) U.S. Cl. CPC A61M 1/362; A61M 1/3496; A61M 2202/0464; A61M 2202/07

(57) ABSTRACT

An embodiment provides a method for treating a body fluid, including: removing the body fluid from a patient; applying a treatment to the body fluid, wherein the treatment comprises an antibody that joins with an antigen in the body fluid to form an antibody-antigen complex; removing the antibody-antigen complex from the body fluid; and returning the body fluid to the patient. Other aspects are described and claimed.

### CROSS REFERENCE TO RELATED APPLICATION

This application claims priority to U.S. Provisional Patent Application Ser. No. 62/989,981, filed on Mar. 16, 2020, and entitled “METHOD FOR TREATING AND CURING COVID-19 INFECTION,” the contents of which are incorporated by reference herein.

### FIELD

This application relates generally to a treatment for Covid-19, and, more particularly, to an extracorporeal methodology for the treatment of Covid-19.

### BACKGROUND

Coronaviruses are a family of viruses that can cause illnesses such as severe acute respiratory syndrome (SARS) and Middle East respiratory syndrome (MERS). A new coronavirus (Covid-19) was identified as the cause of a disease outbreak in China. The virus is presently known as the severe acute respiratory syndrome coronavirus 2 (SARS-CoV-2). The disease it causes is called coronavirus disease 2019 (COVID-19).

Cases of COVID-19 have been reported in multiple countries, where it has caused a great deal of morbidity and mortality, in a worldwide pandemic. The disorder is characterized by shortness of breath, increased mucus production, sore throat, cough, and fever. This may necessitate admission to a hospital, with subsequent admission to an intensive care unit for the respiratory support of the infected patient.

There is a need for treatments for Covid-19, due to the worldwide pandemic of this infection.

### BRIEF SUMMARY

In summary, one embodiment provides a method for treating a body fluid, comprising: removing the body fluid from a patient; applying a treatment to the body fluid, wherein the treatment comprises an antibody that joins with an antigen in the body fluid to form an antibody-antigen complex; removing the antibody-antigen complex from the body fluid; and returning the body fluid to the patient.

Another embodiment provides a device for treating a body fluid extracorporeally, comprising: a first stage including an inlet for the body fluid and at least one exterior wall defining a treatment chamber; a second stage, fluidly connected to the first stage, comprising a removal module and an outlet for the body fluid, wherein the treatment chamber comprises a delivery tube for introducing a treatment into the treatment chamber, wherein the delivery tube comprises a hollow tube including at least one interior wall defining a plurality of holes through which the treatment can be added to the treatment chamber, wherein the treatment is delivered through the hollow tube in counter-current mode with reference to the body fluid; the device being configured to: remove the body fluid from a patient; apply a treatment to the body fluid, wherein the treatment comprises an antibody that joins with an antigen in the body fluid to form an antibody-antigen complex; remove the antibody-antigen complex from the body fluid; and return the body fluid to the patient.

A further embodiment provides a product for treating a body fluid extracorporeally, comprising: a first stage including an inlet for the body fluid and at least one exterior wall defining a treatment chamber; a second stage, fluidly connected to the first stage, comprising a removal module and an outlet for the body fluid, wherein the treatment chamber comprises a delivery tube for introducing a treatment into the treatment chamber, wherein the delivery tube comprises a hollow tube including at least one interior wall defining a plurality of holes through which the treatment can be added to the treatment chamber, wherein the treatment is delivered through the hollow tube in counter-current mode with reference to the body fluid.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example partial cross sectional view of a cylinder and tubing used to deliver a treatment to a bodily fluid.

FIG. 2 illustrates an example a partial cross sectional view showing additional detail of the cylinder and tubing of FIG. 1.

FIG. 3 illustrates an example flow diagram of a method for treatment of Covid-19 using extracorporeal methodology.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

COVID-19 has spread worldwide and become a global pandemic. The loss of life, suffering, and economic struggles have reached all corners of the globe. Symptoms may manifest about 2-14 days after exposure. The symptoms may include fever, chills, cough, shortness of breath, difficulty breathing, fatigue, muscle/body aches, new loss of taste/smell, sore throat, congestion, runny nose, nausea, vomiting, or diarrhea. More severe symptoms may include trouble breathing, persistent pain/pressure in the chest, confusion, inability to wake or stay awake, or bluish lips/face. Some cases may require hospitalization and even intensive care unit healthcare. Because of the novelty of the virus, very few tests exist that are specific for COVID-19. What is needed is a treatment of COVID-19 in a patient.

Coronaviruses are a family of viruses that can cause illnesses such as severe acute respiratory syndrome (SARS) and Middle East respiratory syndrome (MERS). A new coronavirus (Covid-19) was identified as the cause of a disease outbreak in China. The virus is known as the severe acute respiratory syndrome coronavirus 2 (SARS-CoV-2). The disease it causes is called coronavirus disease 2019 (COVID-19).

In a phylogenetic analysis of 103 strains of SARS-CoV-2 from China, two different types of SARS-CoV-2 were identified, designated type L (accounting for 70 percent of the strains) and type S (accounting for 30 percent). The strains in L type, derived from S type, are evolutionarily more aggressive and contagious.

Cases of COVID-19 have been reported in multiple countries, where it has caused a great deal of morbidity and mortality, in a worldwide pandemic. The disorder is characterized by shortness of breath, increased mucus production, sore throat, cough, and fever. This may necessitate admission to a hospital, with subsequent admission to an intensive care unit for the respiratory support of the infected patient. There is therefore a need for treatments to reduce Covid-19 symptomatology in a clinical setting.

Accordingly, an embodiment provides a method for treating COVID-19 extracorpeally, or outside of a patient. In an embodiment, a method for treating a body fluid is disclosed. In an embodiment, a body fluid may be obtained from a patient. In an embodiment, the method may apply a treatment to the body fluid, once it is outside the body of the patient, by introducing an antibody that joins with an antigen in the body fluid to form an antibody-antigen complex. The method may remove the antibody-antigen complex from the body fluid. The targeted antigen may be selected from a group consisting of nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex. Other targets and/or antigens are disclosed. In an embodiment, the method may return the body fluid to the patient.

The present application relates to an article and method of extracorporeal treating a patient's body fluid, for example, blood, and/or CSF (cerebrospinal fluid).

The treatment includes a plurality of stages comprising removing the body fluid from a patient, applying an extracorporeal treatment to the body fluid, and returning the body fluid to the patient.

In the first stage of the treatment, the body fluid (blood and/or CSF) is removed from the patient. A convenient method for removing blood is by using standard venipuncture technique. A convenient method for removing the CSF is by using standard lumbar puncture technique. In the second stage, a treatment is applied to the body fluid (blood and/or CSF). The treatment can include an antibody directed against targeted antigen(s)/TA(s). The third stage comprises returning the body fluid to the patient and can also include removing the treatment from the body fluid so treated.

The method of the present method comprises treating a patient's body fluid extracorporeally with antibody(s) designed to react with particular targeted antigen(s)/TA(s) of Covid-19: nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex.

In an embodiment, the antibody comprises an albumin moiety and targets the removal of the TA(s) from the blood, or body fluid. One removal method includes using dialysis to remove the antibody-antigen complex. Various dialysis methods are known by one skilled in the art are contemplated in this disclosure.

The Covid-19 target antigen(s)/TA(s): nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex, can be identified and differentiated using standard ELISA methodology. Identification can be done before treatment to determine which TAs are present in patient's blood and after treatment to analyze the efficiency of removal of the TA. ELISA (enzyme-linked immunosorbant assay) is a biochemical technique which allows for the detection of an antigen in a sample. In ELISA an antigen is affixed to a surface, and then an antibody is utilized for binding to the antigen. The antibody is linked to an enzyme which enables a color change in the substrate. Other strategies may be employed to validate the level of target antigen(s)/TA(s) in the body fluid before or after treatment: Western blotting technology, UV/Vis spectroscopy, mass spectrometry, and surface plasmon resonance (SPR).

In an embodiment, the methodology described herein may be used to treat other conditions. For example, target antigens may be constructed for many conditions, diseases, infections, or the like. Pathogenic bacteria examples such as, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, Yersinia pseudotuberculosis, or the like may be treated.

An alternative methodology would utilize a designer antibody with an attached macromolecular moiety instead of an albumin moiety. The macromolecular moiety, attached to the antibody, would be 1.000 mm to 0.00001 mm in diameter. The antibody-macromolecular moiety-targeted antigen complex would then be blocked from reentering the patient's blood, by using a series of microscreens which contain openings with a diameter 50.00000% to 99.99999% less than the diameter of the designer antibody-macromolecular moiety. The microscreen opening(s) must have a diameter of at least 25 microns to allow for the passage and return to circulation of the non-pathology-inducing body fluid constituents.

Alternatively, the target antigen(s)/TA(s): nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex, may be captured by using antibody microarrays which contain antibodies to the target antigen(s). The antibody microarrays are composed of millions of identical monoclonal antibodies attached at high density on glass or plastic slides. After sufficient extracorporeal exposure of the TA(s) to the antibody microarrays, the antibody microarrays-TA(s) may be disposed of, using standard medical practice.

Another alternative methodology comprises removing the targeted antigen(s)/TA(s): nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, nsp10-nsp16 complex, from the body fluid by using a designer antibody containing an iron (Fe) moiety. This will then create an Fe-Antibody-Antigen complex. This iron containing complex may then be efficaciously removed using a strong, localized magnetic force field.

In an embodiment, a treatment is applied to a body fluid extracorporeally. The treatment comprises exposing the body fluid to a tagged antibody generated to bind specific targeted pathogenic antigens (TPAs) of the Covid-19 virus, or other target, such as those described above. During this treatment the conjugated antibody(s) and the targeted pathogen antigen form antibody complexes. A method for enhancing radiofrequency (RF) absorption includes providing targeted RF enhancers, such as antibodies with an attached RF absorption enhancer, such as, for example metal particles. The antibodies target and bind to the Covid-19 virion. Binding RF enhancing particles to the antibodies (and other carriers having at least one targeting moiety) permits the injection of the antibodies (and other carriers having at least one targeting moiety) into the extracorporeal target solution. The RF enhancers induce the absorption of energy in the antibody-RF enhancing moiety complex. In addition, a combination of antibodies (and other carriers having at least one targeting moiety bound to different metals (and other RF absorbing particles) can be used allowing for variations in the RF absorption characteristics in the extracorporeal target area. The energy of the emitted radiofrequency (RF) annihilates the antibody-RF enhancing moiety complex, thereby destroying its disease-causing potential. The entire system is monitored and controlled utilizing a computer, in real time, utilizing time units of 1 millisecond or less during the entire procedure. Persons having ordinary skill in art will recognize that the steps described above can be performed on various devices/machines. This disclosure contemplates all known devices/machine that can perform the steps described in the above illustrative example.

A second stage substantially eliminates, through a high-energy radiofrequency emissive source targeting and annihilating, the antibody-RF enhancing moiety complex in the body fluid. A method for killing the Covid-19 virus, or other virus or bacteria, is by introducing into the extracorporeal patient body fluid (blood or CSF) RF absorption enhancers capable of selectively binding to the target virions and further capable of generating sufficient heat to kill or damage the bound target antibody-virion complexes by heat generated solely by the application of an RF field generated by an RF signal between a transmission head and a reception head.

Alternatively, immunoaffinity chromatography may be employed in which the heterogeneous group of molecules in the body fluid will undergo a purification process. There will be an entrapment on a solid or stationary phase or medium. Only the targeted antigens (TAs) will be trapped using immunoaffinity chromatography. A solid medium can be removed from the mixture, washed, and the TA(s) may then be released from the entrapment through elution.

Alternatively, gel filtration chromatography may be utilized in which the body fluid is used to transport the sample through a size exclusion column that will be used to separate the target antigen(s)/TA(s) by size and molecular weight.

Another alternative methodology would utilize a molecular weight cut-off filtration. Molecular weight cut-off filtration refers to the molecular weight at which at least 80% of the target antigen(s)/TA(s) is prohibited from membrane diffusion.

The method comprises at least three stages including a first stage, a second stage and a third stage. The first stage comprises removing body fluid from a patient. The second stage treats the blood and/or CSF as discussed throughout this application. The thirds stage returns the treated body fluid to the patient after having achieved the physical removal of the targeted antigen(s): nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, nsp10-nsp16 complex.

The treatment removes of the targeted antigen(s) from the body fluid. The cleansed body fluid can then be returned to the patient, for example by using the same catheter that was originally used in removing the body fluid. In one embodiment, the treatment of blood comprises removing 25 ml to 500 ml of blood from a patient, and then applying the treatment to the blood before returning it to the patient. The frequency of such treatments would depend upon an analysis of the underlying symptomatology and pathology of the patient.

An alternative method utilizes a device that comprises two-stages. The first stage includes an inlet for body fluid and at least one exterior wall defining a treatment chamber that is fluidly connected to a second stage. The second stage comprises a removal module and an outlet for the body fluid. In embodiments, the removal module is selected from a group comprising a mechanical filter, a chemical filter, a dialysis machine, a molecular filter, molecular adsorbent recirculating system (MARS), a plasmapheresis unit, or combinations thereof.

The method includes removing body fluid (blood and/or CSF) from a patient in a first stage, treating the body fluid to obtain a reduction in the target antigen(s)/TA(s), and optionally removing the treatment from the body fluid in a second stage, and returning the body fluid to the patient in a third stage. The body fluid (blood and/or CSF) can be removed from the patient using any convenient method, including standard venipuncture procedure and/or lumbar puncture. The second stage can include sequentially passing the extracorporeal body fluid through a treatment chamber and a removal module.

The second stage applies a treatment to the blood and/or CSF, which can include introducing a designer antibody that joins with a targeted antigen (TA) in the bodily fluid to form an antibody-antigen complex. The antibody-antigen complex can be removed from the blood and/or CSF in the removal module. Optionally, the antibody-antigen complex can be conjugated with a second antibody, which is then removed, and the purified body fluid is then returned to the patient.

The device comprises a first stage including an inlet for body fluid (blood and/or CSF) and at least one exterior wall defining a treatment chamber that is fluidly connected to a second stage comprising a removal module and an outlet for the body fluid to be treated. The treatment chamber can include a delivery tube for introducing a treatment into the treatment chamber. In embodiments, the delivery tube comprises a hollow tube including at least one interior wall defining a plurality of holes through which the treatment can be added to the treatment chamber. The treatment can also be delivered through the hollow tube in counter-current mode with reference to the flow of the extracorporeal body fluid. The removal module can be any device capable of removing the antibody-antigen complex. In embodiments, the removal module can be selected from a group comprising a mechanical filter, a chemical filter, a dialysis machine, a molecular filter, molecular adsorbent recirculating system (MARS), a plasmapheresis unit, or combinations thereof.

In an example, the first stage of the device applies a treatment of an antibody with an attached albumin moiety that targets the antigen(s)/TA(s): nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, nsp10-nsp16 complex. The second stage includes substantial removal of the treatment from the extracorporeal body fluid.

The illustrated example embodiments will be best understood by reference to the figures. The following description is intended only by way of example, and simply illustrates certain example embodiments.

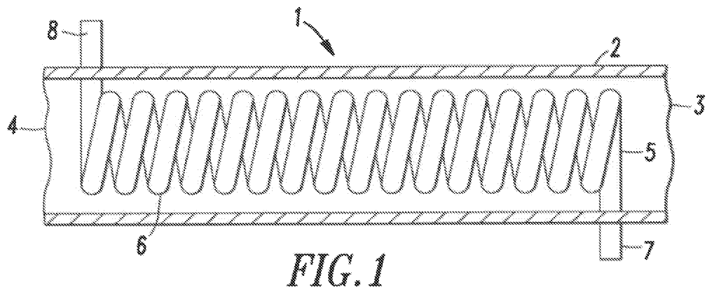

As shown in FIG. 1, the first stage can include an exterior wall 2 defining a treatment chamber 5. The treatment can be applied in the treatment chamber 5. Residence times of the blood to be treated can be altered by changing the dimensions of the treatment chamber or the flow rate of the body fluid through the treatment chamber 5. body fluid to be treated enters the inlet 3, passes through the treatment chamber 5, and exits the outlet 4. In embodiments, the treatment can be applied from a delivery tube 6 located within the treatment chamber 5. An interior wall 9 defines the delivery tube 6. The delivery tube 6 can include at least one lead 7, 8. The lead 7, 8 can deliver the treatment to the treatment chamber 5. Conveniently, the delivery tubes 6 will have a high contact surface area with the body fluid. As shown, the delivery tube 6 comprises a helical coil.

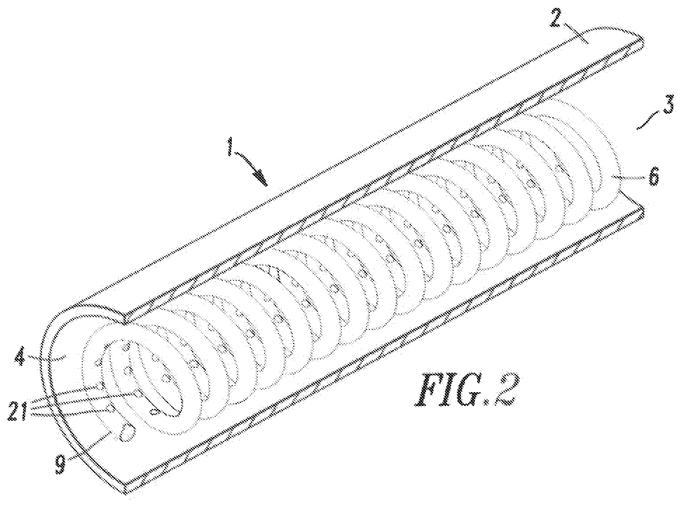

With reference to FIG. 2, the delivery tube 6 can be hollow and the interior wall 9 can define a plurality of holes 21. In this design for example, designer antibodies can be pumped through the delivery tube 6 to affect a desired concentration of designer antibodies in the body fluid (blood and/or CSF). The designer antibodies can perfuse through the holes 21. The delivery tube 6 can include any suitable material including, for example, metal, plastic, ceramic or combinations thereof. The delivery tube 6 can also be rigid or flexible. In one embodiment, the delivery tube 6 is a metal tube perforated with a plurality of holes. Alternatively, the delivery tube 6 can be plastic.

The antibody with attached albumin moiety, targeting the antigen: can be delivered in a concurrent or counter-current mode with reference to the blood and/or CSF. In counter-current mode, the body fluid enters the treatment chamber 5 at the inlet 3. The designer antibody can enter through a first lead 8 near the outlet 4 of the treatment chamber 5. The body fluid then passes to the outlet 4 and the designer antibodies pass to the second lead 7 near the inlet 3. The removal module of the second stage substantially removes the designer antibodies-antigen molecular compound from the blood and/or CSF.

The second stage can include a filter, such as a dialysis machine, which is known to one skilled in the art. The second stage can include a molecular filter. For example, molecular adsorbents recirculating system (MARS), which may be compatible and/or synergistic with dialysis equipment. MARS technology can be used to remove small to average sized molecules from the blood and/or CSF. Artificial liver filtration presently uses this technique.

The methodology can include a plurality of steps for removing the targeted antigen(s)/TA(s): nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex. A first step can include directing a first antibody against the targeted antigen. A second step can include a second antibody. The second antibody can be conjugated with albumin, or alternatively a moiety which allows for efficacious dialysis. The second antibody or antibody-albumen complex combines with the first antibody forming an antibody-antibody-moiety complex. A third step is then utilized to remove the complex from the blood and/or CSF. This removal is enabled by using dialysis and/or MARS. The purified blood can then be returned to the patient.

In practice, a portion of the purified body fluid can be tested to ensure a sufficient portion of the targeted antigen(s)/TA(s): nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex has been successfully removed from the body fluid using methods discussed throughout this application. Testing can determine the length of treatment and evaluate the efficacy of the sequential dialysis methodology in removing the targeted antigens. Body fluid with an unacceptably high concentration of complex remaining can then be re-treated before returning the body fluid to the patient.

In embodiments, the second stage treating the body fluid to remove the antibody-moiety-targeted antigen complex by various techniques including, for example, filtering based on molecular size, protein binding, solubility, chemical reactivity, and combinations thereof. For example, a filter can include a molecular sieve, such as zeolite, or porous membranes that capture complexes comprising molecules above a certain size. Membranes can comprise polyacrylonitrile, polysulfone, polyamides, cellulose, cellulose acetate, polyacrylates, polymethylmethacrylates, and combinations thereof. Increasing the flow rate or diasylate flow rate can increase the rate of removal of the antibody with attached albumin moiety targeting the antigen(s)/TA(s): nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex.

Additional embodiments can include continuous renal replacement therapy (CRRT) which can remove large quantities of filterable molecules from the extracorporeal body fluid. CRRT would be particularly useful for molecular compounds that are not strongly bound to plasma proteins. Categories of CRRT include continuous arteriovenous hemofiltration, continuous venovenous hemofiltration, continuous arteriovenous hemodiafiltration, slow continuous filtration, continuous arteriovenous high-flux hemodialysis, and continuous venovenous high flux hemodialysis.

The sieving coefficient (SC) is the ratio of the molecular concentration in the filtrate to the incoming bodily fluid. A SC close to zero implies that the moiety antibody-targeted antigen complex will not pass through the filter. A filtration rate of 10 ml per minute is generally satisfactory. Other methods of increasing the removability of the moiety-antibody-targeted antigen include the use of temporary acidification of the bodily fluid using organic acids to compete with protein binding sites.

Some embodiments are outlined in the following:

A method for treating a body fluid comprising: a first stage including removing the body fluid from a patient; a second stage including applying a treatment to the body fluid and removing the treatment from the body fluid; and a third stage including returning the body fluid to the patient.

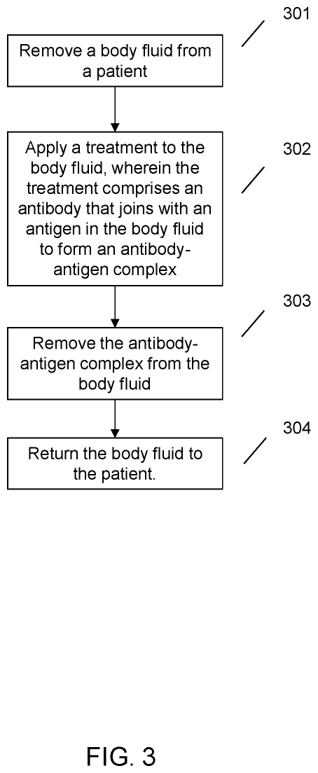

Referring to FIG. 3, an example method is illustrated. A method for treating a body fluid comprising: a first stage including removing the body fluid from a patient at 301; a second stage including applying a treatment to the body fluid by introducing an antibody that joins with an antigen in the body fluid to form an antibody-antigen complex at 302, and removing the antibody-antigen complex from the body fluid at 303; and a third stage including returning the body fluid to the patient at 304.

A method for treating a body fluid comprising: a first stage including removing the body fluid from a patient; a second stage including applying a treatment to the body fluid by introducing an antibody that joins with an antigen in the body fluid to form an antibody-antigen complex, and removing the antibody-antigen complex from the body fluid; wherein the targeted antigen is selected from a group consisting of nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex; and a third stage including returning the body fluid to the patient.

A method for treating a body fluid comprising: a first stage including removing the body fluid from a patient; a second stage including applying a treatment to the body fluid introducing a targeted antibody that joins with an antigen in the body fluid to form an antibody-antigen complex; and conjugating the antibody-antigen complex with a second antibody comprising a moiety that increases the efficacy of removal, thereby forming an antibody-moiety-antigen complex; and a third stage including returning the body fluid to the patient

A method for treating a body fluid comprising: a first stage including removing the body fluid from a patient; a second stage including applying a treatment to the body fluid by introducing an antibody that joins with an antigen in the body fluid to form an antibody-antigen complex, and removing the complex from the body fluid; wherein the targeted antigen is selected from a group consisting of nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, nsp10-nsp16 complex; and a third stage including returning the body fluid to the patient; wherein the method includes determining the efficacy of treatment by testing the body fluid after the treatment and before returning the body fluid to the patient.

A device for treating extracorporeal body fluid comprising a first stage including an inlet for the body fluid and at least one exterior wall defining a treatment chamber that is fluidly connected to a second stage comprising a removal module and an outlet for the blood or CSF, wherein the treatment chamber comprises a delivery tube for introducing a treatment into the treatment chamber, wherein delivery tube comprises a hollow tube including at least one interior wall defining a plurality of holes through which the treatment can be added to the treatment chamber.

A device for treating extracorporeal body fluid comprising a first stage including an inlet for the body fluid and at least one exterior wall defining a treatment chamber that is fluidly connected to a second stage comprising a removal module and an outlet for the blood or CSF, wherein the treatment is delivered through the hollow tube in counter-current mode with reference to the body fluid.

Also a device for treating extracorporeal body fluid comprising a first stage including an inlet for the body fluid and at least one exterior wall defining a treatment chamber that is fluidly connected to a second stage comprising a removal module and an outlet for the blood and/or CSF, wherein the removal module is selected from a group comprising a mechanical filter, a chemical filter, a dialysis machine, a molecular filter, molecular adsorbent recirculating system (MARS), a plasmapheresis unit, or combinations thereof.

Unless otherwise indicated, all numbers expressing quantities of ingredients, properties such as molecular weight, reaction conditions, and so forth used in the specification and claims are to be understood as being modified in all instances by the term “about.

Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

While the invention has been particularly shown and described with reference to a preferred embodiment, it will be understood by those skilled in the art that various changes in form and detail may be made therein without departing from the spirit and scope of the invention. All documents, books, manuals, papers, patents, published patent applications, guides, abstracts, and other references cited herein are incorporated by reference in their entirety.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

It can be appreciated from the foregoing that electronic components of one or more systems or devices may include, but are not limited to, at least one processing unit, a memory, and a communication bus or communication means that couples various components including the memory to the processing unit(s). A system or device may include or have access to a variety of device readable media. System memory may include device readable storage media in the form of volatile and/or nonvolatile memory such as read only memory (ROM) and/or random access memory (RAM). By way of example, and not limitation, system memory may also include an operating system, application programs, other program modules, and program data.

Embodiments may be implemented as an instrument, system, method or program product. Accordingly, an embodiment may take the form of an entirely hardware embodiment, or an embodiment including software (including firmware, resident software, micro-code, etc.) that may all generally be referred to herein as a “circuit,” “module” or “system.” Furthermore, embodiments may take the form of a program product embodied in at least one device readable medium having device readable program code embodied thereon.

A combination of device readable storage medium(s) may be utilized. In the context of this document, a device readable storage medium (“storage medium”) may be any tangible, non-signal medium that can contain or store a program comprised of program code configured for use by or in connection with an instruction execution system, apparatus, or device. For the purpose of this disclosure, a storage medium or device is to be construed as non-transitory, i.e., not inclusive of signals or propagating media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and products according to various example embodiments. It will be understood that the actions and functionality may be implemented at least in part by program instructions. These program instructions may be provided to a processor of a device, e.g., a hand held measurement device, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for treating a body fluid, comprising:
removing the body fluid from a patient;
applying a treatment to the body fluid, wherein the treatment comprises an antibody that joins with an antigen in the body fluid to form an antibody-antigen complex;
removing the antibody-antigen complex from the body fluid; and
returning the body fluid to the patient.

2. The method of claim 1, wherein the antigen is selected from the group consisting of: nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex.

3. The method of claim 1, wherein the antigen is selected to target at least one pathogenic bacteria.

4. The method of claim 1, further comprising conjugating the antibody-antigen complex with a second antibody.

5. The method of claim 4, wherein the second antibody comprises a moiety that increases the efficacy of removal, thereby forming an antibody-moiety-antigen complex.

6. The method of claim 5, wherein the moiety comprises an iron moiety.

7. The method of claim 1, further comprising determining an efficacy of treatment based on testing the body fluid after the treating the body fluid and before returning the body fluid to the patient.

8. The method of claim 1, wherein the body fluid is selected from the group consisting of: blood and cerebrospinal fluid.

9. The method of claim 1, wherein the removal is selected from a group consisting of: a mechanical filter, a chemical filter, a dialysis machine, a molecular filter, molecular adsorbent recirculating system (MARS), and a plasmapheresis unit.

10. The method of claim 1, further comprising applying a radiofrequency emission to the antigen and the body fluid.

11. A device for treating a body fluid extracorporeally, comprising:
a first stage including an inlet for the body fluid and at least one exterior wall defining a treatment chamber;
a second stage, fluidly connected to the first stage, comprising a removal module and an outlet for the body fluid, wherein the treatment chamber comprises a delivery tube for introducing a treatment into the treatment chamber, wherein the delivery tube comprises a hollow tube including at least one interior wall defining a plurality of holes through which the treatment can be added to the treatment chamber, wherein the treatment is delivered through the hollow tube in counter-current mode with reference to the body fluid;
the device being configured to:
remove the body fluid from a patient;
apply a treatment to the body fluid, wherein the treatment comprises an antibody that joins with an antigen in the body fluid to form an antibody-antigen complex;
remove the antibody-antigen complex from the body fluid; and
return the body fluid to the patient.

12. The device of claim 11, wherein the antigen is selected from the group consisting of: nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex.

13. The device of claim 11, wherein the antigen is selected to target at least one pathogenic bacteria.

14. The device of claim 11, further comprising conjugating the antibody-antigen complex with a second antibody.

15. The device of claim 14, wherein the second antibody comprises a moiety that increases the efficacy of removal, thereby forming an antibody-moiety-antigen complex.

16. The device of claim 15, wherein the moiety comprises an iron moiety.

17. The device of claim 11, further comprising determining an efficacy of treatment based on testing the body fluid after the treating the body fluid and before returning the body fluid to the patient.

18. The device of claim 11, wherein the body fluid is selected from the group consisting of: blood and cerebrospinal fluid.

19. The device of claim 11, wherein the removal is selected from a group consisting of: a mechanical filter, a chemical filter, a dialysis machine, a molecular filter, molecular adsorbent recirculating system (MARS), and a plasmapheresis unit.

20. A product for treating a body fluid extracorporeally, comprising:
a first stage including an inlet for the body fluid and at least one exterior wall defining a treatment chamber;
a second stage, fluidly connected to the first stage, comprising a removal module and an outlet for the body fluid, wherein the treatment chamber comprises a delivery tube for introducing a treatment into the treatment chamber, wherein the delivery tube comprises a hollow tube including at least one interior wall defining a plurality of holes through which the treatment can be added to the treatment chamber, wherein the treatment is delivered through the hollow tube in counter-current mode with reference to the body fluid.
